(19) United States
(12) Patent Application Publication
(10) Pub. No.: US 2009/0155883 A1
(43) Pub. Date: Jun. 18, 2009

(54) Scientifically Modulated And Reprogrammed Treatment (SMART) Virus Technology Intended to Neutralize the Human Immunodeficiency Virus

(76) Inventors: Lane Bernard SCHEIBER, Annandale, VA (US); Lane Bernard SCHEIBER, II, Grosse Ile, MI (US)

Correspondence Address: LANE B. SCHEIBER, 7864 OAK RIVER DRIVE, GROSS ILE, MI, 48138, US

(21) Appl. No.: 11/957,437

(22) Filed: Dec. 15, 2007

(51) Int. Cl. C12N 7/00

(52) U.S. Cl. 435/235.1

(57) ABSTRACT

The concept is to combat one of the deadliest infectious diseases known by fighting fire with fire. Scientifically Modulated And Reprogrammed Treatment (SMART) Virus technology is intended to neutralize the Human Immunodeficiency Virus. The SMART Virus carrying combinations of CD4, CCR5 and CXCR4 cell-surface receptors is capable of engaging the HIV's gp 120 and gp 41 exterior probes in an identical manner as would HIV's host, a T-Helper cell. When HIV encounters a SMART Virus the HIV virion would either adhere to the SMART Virus unable to further migrate in search of a T-Helper cell, harmlessly eject its RNA genetic payload or inject its RNA genome into the SMART Virus. Given HIV engaged a SMART Virus rather than a T-Helper cell, the HIV RNA genome becomes unable to infect a T-Helper cell, therefore the threat of Acquired Immunodeficiency Syndrome caused by HIV is averted.

### BACKGROUND OF THE INVENTION

1. Field of the Invention

This invention relates to any medical device intended to physically interact directly with the Human Immunodeficiency Virus (HIV) or other virus to neutralize the capacity of the virus to properly infect its target host cell.

2. Description of the Background Art

The Human Immunodeficiency Virus (HIV), which is responsible for Acquired Immunodeficiency Disease Syndrome (AIDS), threatens the lives of approximately 170 millions of people worldwide. There are different strains of HIV that exist around the world. Most predominantly HIV-1 exists worldwide and HIV-2 is generally found in Western Africa, the western coastal regions of India and in Europe. Amongst HIV-1 and HIV-2 they can be further subdivided into different strains including an ‘R5’ strain which uses a CCR5 cell-surface receptor on a T-Helper cell to identify its host and an ‘X4’ strain which uses a CXCR4 cell-surface receptor located on a T-Helper cell to identify its host. The approach to controlling this disease has been the application of drugs directed at interfering with the replication process, in an attempt to slow down the rate of replication of the virus. Millions of people continue to die and the virus continues to pose an escalating threat despite current treatment strategies. The virus is generally communicated between individuals by contact with body fluids carrying intact HIV.

Though there are recognized differences between HIV-1 and HIV-2, for purposes of further discussion the term ‘HIV’ will refer to both HIV-1 and HIV-2, unless otherwise noted. HIV is a retrovirus with its genetic material in the form of two identical copies of a positive sense single stranded ribonucleic acid (RNA) molecule, each approximately 9500 nucleotides long. HIV is approximately 50 nm in diameter, about one seventieth the size of a white cell carrying the marker Cluster Designation 4 (CD4) exterior cell-surface receptor.

A eukaryote cell is a nucleated cell. Animal cells generally are comprised of a cell membrane, cytoplasm, a nucleus and organelles. The cell membrane consists of a lipid bilayer where two layers of lipid molecules oriented with their polar ends pointed outside of the membrane and their nonpolar ends points toward the inside of the membrane. Polarized ends of the lipid molecules are hydrophobic, therefore the lipid bilayer functions to control the movement of water, nutrients and hormones in and out of the cell. A variety of receptors affixed to the exterior of the lipid bilayer membrane assist in the cell communicating with its environment. The cytoplasm inside a cell is comprised of amino acids and nutrients, and forms the interior fluid matrix of the cell. The nucleus is surrounded by a double membrane and contains the majority of the cell's genetic material. Organelles are structures generally found in the cytoplasm that perform specialized functions of the cells. Organelles found inside a cell may include the mitochondria, endoplasmic reticulum, Golgi complex, lysosomes, vacuoles.

Genetic material in a eukaryote is generally in the form of deoxyribonucleic acid (DNA) with the majority located in the nucleus of the cell, but DNA may also be found in the mitochondria of cells. By the process of transcription, a section of the DNA is read by a polymerase and a molecule of ribonucleic acid (RNA) is generated. DNA is comprised of sections of combinations of four nucleotides: adenine, cytosine, guanine, and thymine. When two strands of nucleotides are arranged together, such as in the double helix configuration of chromosomal DNA, adenine on one strand is always matched to thymine in the opposing strand and cytosine on one strand is always matched to guanine in the opposing strand. RNAs that are generated are usually single stranded chains of nucleotides, constructed of similar adenine, cytosine and guanine nucleotides as DNA, but instead of ‘thymine’, RNAs are constructed with the nucleotide ‘uracil’. RNAs are generally divided into three categories including messenger RNA (mRNA), ribosomal RNA (rRNA) and transfer RNA (tRNA). Messenger RNA are considered positive sense and interact with ribosomes to generate protein molecules. Ribosomes read the code physically built into the messenger RNAs, and with the aid of rRNA and tRNA, generate protein molecules by bonding together amino acids in linear configurations as directed by the code on the messenger RNA.

HIV is approximately spherical in shape and comprised of an outer lipid bilayer envelope, a matrix protein, a capsid, two strands of RNA, nucleocapsid protein and proteins to assist in the replication process. The virus's core or capsid is icoshedral in shape and acts as a protective shell to carry the genetic payload. The capsid is comprised of numerous copies of the capsid protein (p24), the number and arrangement of the capsid proteins determines the overall dimensions of the capsid shell; HIV uses approximately 2,000 p24 to construct its capsid. The capsid carries the two single strands of RNA each containing a copy of the virus's nine genes, the nucleocapsid protein, reverse transcriptase, protease and integrase. The nucleocapsid protein causes the RNA to coil up so that it can fit inside the capsid. The protein matrix consisting of protein 17 (p17) covers the capsid. The HIV envelope is derived from the plasma membrane as the virus buds or pushes through the host cell's plasma membrane as it exits and migrates from the host cell. Anchored in and projecting out from the HIV's lipid bilayer outer membrane, otherwise referred to as an envelope, are exterior probes well known to the medical and scientific community as glycoprotein 120 (gp 120) and glycoprotein 41 (gp 41). The term glycoprotein refers to a protein with a carbohydrate attached. The gp 41 probe is anchored to the outer envelope and is in close proximity to the gp 120 probe. The probes can be found arranged together into protein complexes, which may contain up to three gp 120 probes and three gp 41 probes. Protein complexes have been described as ‘spikes’. It has been reported that an HIV outer envelope may project from ten to seventy-two said spikes.

Viruses are obligate intracellular parasites designed to infect cells often with great specificity to a particular cell type it uses as a host. Viruses do not carry out any biologically active processes on their own when outside a host cell. A virus requires a host in order to reproduce itself. Viruses circulate the environment without the need for nutrition or energy production through respiration. Viruses are in essence a vehicle that carries the genetic programming instructions necessary to cause an appropriate host cell to generate identical copies of the same virus. Some viruses do introduce to their host cells programming instructions that result in toxic effects to the body that has been infected by the virus. HIV transits the environment at large with its surface probes seeking to engage its host, a human T-Helper cell. Human T-Helper cells express a number of receptors on their outer plasma membrane including Cluster Designation 4 (CD4), Chemotactic Chemokine Receptor 5 (CCR5) and CX Chemokine Receptor 4 (CXCR4).

HIV utilizes the Human T-Helper cell as its host for the purpose of replicating copies of itself. To initiate its reproductive-cycle, the gp 120 probe on a HIV virion makes initial contact with a T-Helper cell's CD4 cell-surface receptor. Virion is a term that refers to a complete structure of a virus as it exists outside of a host cell. Following the engagement of the gp 120 probe with the CD 4 cell-surface receptor, the gp 120 alters its configuration to allow the HIV gp 41 probe to engage a second receptor on the surface of the T-Helper cell, either a CCR5 cell-surface receptor or a CXCR4 cell-surface receptor. Once the HIV virion's gp 120 probe has successfully engaged a T-Helper cell's CD4 and the HIV's gp 41 probe has successfully engaged the T-Helper cell's CCR5 or CXCR4 cell-surface receptor, then the HIV virion is able to transfer its capsid containing the two strands of ribonucleic acid (RNA) and the support proteins including as reverse transcriptase, protease, and integrase into the T-Helper cell. Once the capsid has gained access to the interior of the T-Helper cell, utilizing the transferred HIV enzyme ‘reverse transcriptase’, the RNA molecules undergo reverse transcription to deoxyribonucleic acid (DNA). Protease helps modify HIV's genome. Aided by the integrase, the virus's RNA transcribed into DNA migrates to the T-Helper cell's nucleus and is inserted into the T-Helper cell's native DNA. The HIV genetic material then redirects the resources of the T-Helper cell to facilitate the manufacture of copies of HIV.

Most predominantly HIV-1 exists worldwide and HIV-2 is generally found in Western Africa, the western coastal regions of India and Europe. Amongst HIV-1 and HIV-2 that use CD4 as the initial cell-surface receptor to gain entry into a T-Helper cell, they can be further divided by an ‘R5’ strain which uses a CCR5 cell-surface receptor on a T-Helper cell to identify its host; an ‘X4’ strain which uses a CXCR4 cell-surface receptor located on a T-Helper cell to identify its host. It is also believed at least one strain of HIV-2 may infect a T-Helper cell without engaging a CD4 cell-surface receptor, but uses either a CCR5 or a CXCR4 cell-surface receptor on a T-Helper cell host. There has also been identified at least one strain of HIV-2 believed not utilize the CD4, CCR5 or the CXCR4 cell-surface receptors to engage a T-Helper cell host, the mode of entry unknown at this time.

Naturally occurring T-Helper cells, not infected with HIV, help orchestrate the human body's immune response to infectious agents that threaten the health and integrity of the body. The HIV virus, by taking control and altering the function of the T-Helper cells in the body, creates a state of ill health. By redirecting the T-Helper cell's function to produce copies of the HIV virus rather than coordinate appropriate immune responses against potentially infectious agents leaves the body as a whole vulnerable to attack by other infectious agents that can do harm to the tissues of the body. In addition, the HIV genome carries a ‘nef’ gene. Once the HIV's DNA is inserted into the host cell's native DNA, the nef gene provides instructions for a Fas ligand (FasL) cell-surface marker to be manufactured and expressed on the surface of the infected T-Helper cell. Noninfected T-Helper cells (meaning not infected with HIV) express a Fas cell-surface marker. When a HIV infected T-Helper cell expressing the Fas ligand (FasL) cell-surface marker encounters a Fas cell-surface marker (also referred to as CD95 cell-surface receptor) on a noninfected T-Helper cell, a lethal signal is passed to the noninfected t-Helper cell. When the FasL cell-surface marker engages a Fas cell-surface marker, the process of apoptosis is triggered in the noninfected T-Helper cell. Apoptosis is the natural process of programmed cell death. By triggering apoptosis in noninfected T-Helper cells, HIV infected T-Helper cells are capable of killing the noninfected T-Helper cells they encounter. The clinical ramifications of Acquired Immunodeficiency Syndrome (AIDS) occur when the number of noninfected T-Helper cells declines to the point the immune system is unable to defend the body as a whole from dangerous infectious agents.

The Scientifically Modulated And Reprogrammed Treatment (SMART) Virus represents a surrogate target for HIV. SMART viruses would engage HIV directly to negate the infectivity of HIV and therefore neutralize the threat of HIV. It appears the HIV infected T-Helper cells pose the threat to a body's immune system by terminating noninfected T-Helper cells, which a critical number are needed to defend the body from infectious agents. Since the clinical characteristics of AIDS appears to coincide more with the decline of non-infected T-Helper cells below a critical number and not necessarily with the number of HIV infected T-Helper cells, controlling the population of HIV infected T-Helper cells or ridding the environment of HIV infected T-Helper cells would be a successful approach to managing AIDS.

### BRIEF SUMMARY OF THE INVENTION

A Scientifically Modulated And Reprogrammed Treatment (SMART) Virus is comprised of an inner capsid similar to the naturally occurring HIV icoshedral capsid. The SMART Virus capsid would be encapsulated with a protein matrix similar to the protein matrix that encapsulates the HIV's capsid. A lipid bilayer envelope would then encapsulate the matrix protein coat and capsid similar to the lipid bilayer than encapsulates the matrix protein of HIV. Up to three different types of cell-surface receptors are mounted on the outside of the outer envelope, referred to as: CD 4, CCR5 and CXCR4. The three cell-surface receptors CD 4, CCR5 and CXCR4 are identical in construction, arrangement and function to the CD 4, CCR5 and CXCR4 receptors that appear naturally on the surface of noninfected human T-Helper cells. The capsid of the therapeutic version of the SMART Virus would carry either no genetic payload or would carry RNA molecules that would not be capable of replicating the virus-like structure in the natural environment. The amount of genetic payload or filler would relate to the intended stability of the SMART virus once released into the environment. SMART viruses are intended to act as surrogate T-Helper cells by mimicking the T-Helper cell target and engaging HIV's external surface probes in a manner to negate HIV's capability to infect T-Helper cells.

### DETAILED DESCRIPTION

Scientifically Modulated And Reprogrammed Treatment (SMART) Virus technology is intended to neutralize the Human Immunodeficiency Virus wherever the virus might be found. SMART Virus technology is comprised of a lipid bilayer envelope identical in construct to the naturally occurring HIV outer envelope, and mounted to the SMART virus's outer envelope are up to three different types of cell-surface receptors referred to as Cluster Designation 4 (CD4), Chemotactic Chemokine Receptor 5 (CCR5) and CX Chemokine Receptor 4 (CXCR4). The three cell-surface receptors, the CD 4 receptor, the CCR5 receptor and the CXCR4 receptor are well known to the medical and scientific community due to the fact they appear naturally on the surface of the Human T-Helper cell. Within the SMART virus's outer envelope are matrix protein and a capsid to act as the interior conformational structure to provide the appropriate size and shape to the outer envelope as needed. The size of the SMART virus ranges from 7 nanometer (nm) in width (the diameter of HIV is approximately 50 nm) to a diameter in the order of the size of a naturally occurring noninfected T-Helper cell, and up to the diameter of one meter. T-Helper cells are mobile structures and constantly alter their shape and size, but are approximately 3500 nm in diameter. The range in size of the SMART Virus is dependent upon the type of application for which the SMART Virus is required to be utilized.

To initiate its natural reproductive cycle, HIV engages a T-Helper cell by a glycoprotein 120 (gp 120) probe engaging a T-Helper cell's CD4 exterior cell-surface receptor; once this occurs HIV's glycoprotein 41 (gp 41) probe engages the T-Helper cell's CCR5 or CXCR4 exterior cell-surface receptor. The SMART Virus, carrying CD 4, CCR5 and CXCR4 surface receptors, is capable of traveling anywhere HIV virions are likely to travel and engage HIV. When HIV encounters a SMART Virus, the HIV's gp 120 probe would engage the SMART Virus's CD 4 exterior cell-surface receptor, then the HIV's gp 41 probe would engage either the CCR5 or the CXCR4 exterior cell-surface receptor on the SMART Virus. Once HIV's gp 120 and gp 41 probes have engaged their respective receptors on the SMART Virus, HIV would either eject into the surrounding environment the capsid carrying the RNA payload or HIV would inject its capsid and RNA genetic payload into the SMART Virus or the HIV would permanently adhere to the SMART Virus making the HIV virion incapable of being able to interact and infect a T-Helper cell. Since HIV engaged a SMART Virus rather than a T-Helper cell, the HIV RNA genome is rendered harmless and the threat of HIV's infectivity is effectively neutralized. In the event HIV is unable to create additional copies of itself and unable to further threaten T-Helper cells, the threat of Acquired Immunodeficiency Disease Syndrome is successfully neutralized.

SMART Virus technology can be used as a cleaning device to neutralize and rid a surface or a fluid environment of HIV. Since the SMART virus is similar in construction to HIV, the SMART Virus is intended to traverse and survive in any environment where HIV might exist. SMART Virus is intended to engage HIV where ever it may exist.

HIV utilizes T-Helper cells as a natural factory for generating copies of HIV. HIV utilizes a fusion technique where the virion envelope fuses with the cellular membrane of the host cell and directly releases the capsid containing the RNA genome and replicating enzymes into the cytoplasm of the host cell. HIV utilizes enzymes created by its own genome and enzymes native to the T-Helper cell to generate the proteins it requires to construct copies of HIV. Once the appropriate copies of the RNA, nucleocapsid protein, integrase, protease, reverse transcriptase enzyme, capsid proteins, matrix protein and external probes have been manufactured and collected together, the capsid carrying the RNA genetic payload is enveloped by the matrix protein and pushes through the host cell's plasma membrane in a process called budding. The HIV copy becomes encapsulated in an envelope comprised of a lipid bilayer as it separates from the host cell and becomes an independent entity termed a virion. Probes stick out through the lipid bilayer envelope of the virion to seek the receptors located on an appropriate host cell.

To produce copies of the SMART Virus a T-Helper cell can be utilized, bacteria with a lipid bilayer membrane could be utilized or other appropriate host cell could be utilized. The technology to manufacture viruses carrying a therapeutic DNA gene has already been worked out and implemented. The process to generate a medically therapeutic virus to target a particular cell and deliver a specific genetic payload to a specific target cell is a matter of placing inside a host cell the appropriate genetic instructions and enzymes to facilitate the host cell to manufacture the intended ‘medically therapeutic’ virus.

In the case of the SMART Virus, the construction of the final medically therapeutic copies of the SMART Virus include the appropriate instructions and biologic machinery necessary to generate the capsid proteins, matrix proteins, external cell-surface receptors C4, CCR5 and CXCR4, any filling material to be placed inside the capsid and stimulate the budding process. The size of the capsid is dependent upon the capsid proteins used to construct the capsid. For descriptive purposes the use of the term ‘capsid’ is interchangeable with the term ‘capsid shell’. Different naturally occurring viruses are constructed with a different size capsid depending upon the number and arrangement of capsid proteins utilized to construct a virus's capsid. The diameter of the SMART Virus is in part dependent upon the number and arrangement of capsid proteins used to construct the capsid. A genetic payload to act as a filler, incapable of stimulating a disease state in any life form, may be required to fill the inside the capsid of some of the larger diameter SMART Viruses in order to support the successful construction of a particular size of SMART Virus and to facilitate the SMART virus remaining sturdy enough and thus intact once released into the environment to enable it to successfully carry out its intended function.

SMART Virus technology can be used as a cleaning device to neutralize and rid a surface or a fluid environment of HIV by one of three manners. HIV's probes gp 120 and gp 41 are intended to engage a native T-Helper cell's CD4, and the CCR5 or the CXCR4 exterior cell-surface receptor. Once HIV's probes have successfully made contact and functionally engaged a T-Helper cell's receptors HIV injects its capsid into the T-Helper cell. The SMART Virus's receptors would function in the identical manner as a T-Helper cell's receptors. The action of a SMART Virus is to attract and engage HIV. Following the SMART Virus's receptors engaging HIV's probes the SMART Virus would neutralize HIV by: (1) adhering to HIV preventing contact of HIV with a T-Helper cell, (2) causing HIV to eject its capsid into the environment outside HIV's envelope making the HIV genetic payload incapable of infecting a T-Helper cell, (3) causing HIV to inject its capsid into the SMART Virus thus capturing the HIV genetic payload and preventing it from infecting a T-Helper cell. Any of these three actions would interfere with HIV's functional capacity to infect a T-Helper cell. If HIV is unable to infect a T-Helper cell, HIV is unable to replicate and it is unable to further influence T-Helper cells to kill noninfected T-Helper cells by the FasL apoptosis triggering mechanism, and therefore the infectious threat HIV poses is effectively neutralized.

The diameter of the SMART virus is between 7 nm in diameter (the size of the diameter of HIV is approximately 50 nm) to a diameter in the order of the size of a naturally occurring noninfected T-Helper cell, to a diameter of one meter. T-Helper cells are mobile and constantly alter their shape and size, but are approximately 3500 nm in diameter. The range in size of the SMART Virus is dependent upon the type of application for which the SMART Virus is required to be utilized. One or more smaller sized SMART Viruses may simply adhere to a single HIV virion, and thus by adhering to the HIV and engaging the virus's probes make the HIV virion incapable of successfully engaging a T-Helper cell. Larger diameter SMART Viruses might engage one or more HIV virions preventing the HIV virions from further migrating through the environment in search of a T-Helper cell to infect. A SMART Virus may engage a HIV virion and by successfully attaching to the virion's probes in the same manner as a T-Helper cell would engage the virion's probes, cause the HIV virion to eject its capsid into the environment outside the virion's outer envelope. SMART Viruses may also engage a HIV virion and by successfully attaching to the virion's probes in the same manner as a T-Helper cell would engage the virion's probes, cause the HIV virion to inject its capsid into the SMART Virus as if the SMART Virus were a T-Helper cell. The diameter of the SMART Virus would depend upon the size of the reservoir the SMART Virus would be intended to contain to collect one or more HIV capsids. The SMART Virus intended to collect more than one HIV capsid may be best designed not to permanently adhere to HIV once it has accepted the HIV's capsid so that it can functionally engage additional HIV virions to collect additional HIV capsids.

### DRAWING

None.

What is claimed:

1) A medical device comprised of lipid bilayer envelope, which affixed to the said exterior envelope are exterior surface receptors intended to attract, functionally engage and adhere to a target virus's exterior probes with the intention of neutralizing the infectious threat of the target virus.

2) A medical device comprised of lipid bilayer envelope, which affixed to the said exterior envelope are exterior surface receptors intended to attract, functionally engage and adhere to a Human Immunodeficiency Virus's exterior probes with the intention of neutralizing the infectious threat of the Human Immunodeficiency Virus.

3) A medical device consisting of a capsid shell comprised of repeating capsid proteins, this capsid shell encapsulated by matrix proteins, further encapsulated by an exterior lipid bilayer envelope, which affixed to the outer envelope are exterior surface receptors intended to attract and functionally engage a target virus's probes with the intention to neutralize the infectious threat of the target virus.

4) A medical device consisting of a capsid shell comprised of repeating capsid proteins, this capsid shell encapsulated by matrix proteins, further encapsulated by an exterior lipid bilayer envelope, which affixed to the outer envelope are exterior surface receptors intended to attract and functionally engage Human Immunodeficiency Virus's probes with the intention to neutralize the infectious threat of the Human Immunodeficiency Virus.

5) A medical device comprised of a lipid bilayer envelope similar in construct to the materials and design of the naturally occurring Human Immunodeficiency Virus with the said envelope having fixed to its exterior, surface receptors constructed in the same physical form and dimensions as generally known and recognized by the medical scientific community as the CD 4 cell-surface receptor and the CCR5 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and a glycoprotein 41 probe.

6) A medical device comprised of a lipid bilayer envelope similar in construct to the materials and design of the naturally occurring Human Immunodeficiency Virus with the said envelope having fixed to its exterior, surface receptors constructed in the same physical form and dimensions as generally known and recognized by the medical scientific community as the CD 4 cell-surface receptor and the CXCR4 cell-surface receptor as generally found on a naturally occurring human T-Helper cell in a manner and design that will attract, functionally engage and may adhere to one or more probes of one or more sets of an HIV's glycoprotein probes, a set consisting of a glycoprotein 120 probe and a glycoprotein 41 probe.

7) A medical device comprised of a lipid bilayer envelope similar in construct to the materials and design of the naturally occurring Human Immunodeficiency Virus with the said envelope having fixed to its exterior, any combination of surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of an HIV's glycoprotein probes, a set consisting of a glycoprotein 120 probe and a glycoprotein 41 probe.

8) A medical device comprised of an inner capsid enveloped by matrix protein, which is further enveloped by a lipid bilayer envelope all three elements similar in construct to the materials and design of the naturally occurring Human Immunodeficiency Virus with the said outer envelope having fixed to its exterior, surface receptors constructed in the same physical form and dimensions as generally known and recognized by the medical scientific community as the CD 4 cell-surface receptor and the CCR5 cell-surface receptor as generally found on a naturally occurring human T-Helper cell in a manner and design that will attract, functionally engage and may adhere to one or more probes of one or more sets of an HIV's glycoprotein probes, a set consisting of a glycoprotein 120 probe and a glycoprotein 41 probe.

9) A medical device comprised of an inner capsid enveloped by matrix protein, which is further enveloped by a lipid bilayer envelope all three elements similar in construct to the materials and design of the naturally occurring Human Immunodeficiency Virus with the said outer envelope having fixed to its exterior, surface receptors constructed in the same physical form and dimensions as generally known and recognized by the medical scientific community as the CD 4 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe.

10) A medical device comprised of an inner capsid enveloped by matrix protein, which is further enveloped by a lipid bilayer envelope all three elements similar in construct to the materials and design of the naturally occurring Human Immunodeficiency Virus with the said outer envelope having fixed to its exterior, any combination of surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of an HIV's glycoprotein probes, a set consisting of a glycoprotein 120 probe and a glycoprotein 41 probe.

11) A medical device of an overall size that ranges from a width of 7 nm to a size in the order of a T-Helper cell approximately 3500 nm in diameter, comprised of an inner capsid enveloped by matrix protein which by this construct determines the size of the device, which is further enveloped by a lipid bilayer envelope, all three elements similar in construct to the materials and design of the naturally occurring Human Immunodeficiency Virus with the exterior envelope having fixed to its exterior, surface receptors constructed in the same physical form and dimensions as generally known and recognized by the medical scientific community as the CD 4 cell-surface receptor and the CCR5 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe.

12) A medical device of an overall size that ranges from a width of 7 nm to a size in the order of a T-Helper cell approximately 3500 nm in diameter, comprised of an inner capsid enveloped by matrix protein which by this construct determines the size of the device, which is further enveloped by a lipid bilayer envelope, all three elements similar in construct to the materials and design of the naturally occurring Human Immunodeficiency Virus with the exterior envelope having fixed to its exterior, surface receptors constructed in the same physical form and dimensions as generally known and recognized by the medical scientific community as the CD 4 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe.

13) A medical device of an overall size that ranges from a width of 7 nm to a size in the order of a T-Helper cell approximately 3500 nm in diameter, comprised of an inner capsid enveloped by matrix protein which by this construct determines the size of the device, which is further enveloped by a lipid bilayer envelope, all three elements similar in construct to the materials and design of the naturally occurring Human Immunodeficiency Virus with the exterior envelope having fixed to its exterior, any combination of surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe.

14) A medical device of an overall size that ranges from a width of 7 nm to a size in the order of a T-Helper cell approximately 3500 nm in diameter, comprised of an inner capsid enveloped by matrix protein, which is further enveloped by a lipid bilayer envelope all three elements similar in construct to the materials and design of the naturally occurring Human Immunodeficiency Virus with the exterior envelope having fixed to its exterior, surface receptors constructed in the same physical form and dimensions as generally known and recognized by the medical scientific community as the CD 4 cell-surface receptor and the CCR5 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe, which carries a genetic payload to act as a filler, incapable of stimulating a disease state in any life form, required to fill the inside of the capsid of the medical device in order to support the successful construction of a particular size of the medical device to facilitate the medical device remaining sturdy enough during production and to insure the medical device will remain intact and functional once administered as a treatment into an environment where HIV may exist.

15) A medical device of an overall size that ranges from a width of 7 nm to a size in the order of a T-Helper cell approximately 3500 nm in diameter, comprised of an inner capsid enveloped by matrix protein, which is further enveloped by a lipid bilayer envelope all three elements similar in construct to the materials and design of the naturally occurring Human Immunodeficiency Virus with the exterior envelope having fixed to its exterior, surface receptors constructed in the same physical form and dimensions as generally known and recognized by the medical scientific community as the CD 4 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe, which carries a genetic payload to act as a filler, incapable of stimulating a disease state in any life form, required to fill the inside of the capsid of the medical device in order to support the successful construction of a particular size of the medical device to facilitate the medical device remaining sturdy enough during production and to insure the medical device will remain intact and functional once administered as a treatment into an environment where HIV may exist.

16) A medical device of an overall size that ranges from a width of 7 nm to a size in the order of a T-Helper cell approximately 3500 nm in diameter, comprised of an inner capsid enveloped by matrix protein, which is further enveloped by a lipid bilayer envelope all three elements similar in construct to the materials and design of the naturally occurring Human Immunodeficiency Virus with the exterior envelope having fixed to its exterior, surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe, which carries a genetic payload to act as a filler, incapable of stimulating a disease state in any life form, required to fill the inside of the capsid of the medical device in order to support the successful construction of a particular size of the medical device to facilitate the medical device remaining sturdy enough during production and to insure the medical device will remain intact and functional once administered as a treatment into an environment where HIV may exist.

17) A medical device of an overall size that ranges from a width of 7 nm to a size in the order of a T-Helper cell approximately 3500 nm in diameter, comprised of an inner capsid enveloped by matrix protein, which is further enveloped by a lipid bilayer envelope all three elements similar in construct to the materials and design of the naturally occurring Human Immunodeficiency Virus with the exterior envelope having fixed to its exterior, any combination of surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe, which carries a genetic payload to act as a filler, incapable of stimulating a disease state in any life form, required to fill the inside of the capsid of the medical device in order to support the successful construction of a particular size of the medical device to facilitate the medical device remaining sturdy enough during production and to insure the medical device will remain intact and functional once administered as a treatment into an environment where HIV may exist.

18) A medical device of an overall size that ranges from a width of 7 nm to a size to a diameter of one meter, comprised of an inner capsid enveloped by matrix protein, which is further enveloped by a lipid bilayer envelope all three elements similar in construct to the materials and design of the naturally occurring Human Immunodeficiency Virus with the exterior envelope having fixed to its exterior, any combination of surface receptors constructed in the same physical form and dimensions as generally known and recognized by the medical scientific community as the CD 4 cell-surface receptor, the CCR5 cell-surface receptor and the CXCR4 cell-surface receptor as generally found on a naturally occurring human T-Helper cell in a manner and design that will attract, functionally engage and may adhere to one or more probes of one or more sets of HIV's glycoprotein probes, a set consisting of a glycoprotein 120 probe and glycoprotein 41 probe, which carries a genetic payload to act as a filler, incapable of stimulating a disease state in any life form, required to fill the inside of the capsid of the medical device in order to support the successful construction of a particular size of the medical device to facilitate the medical device remaining sturdy enough during production and to insure the medical device will remain intact and functional once administered as a treatment into an environment where HIV may exist.
